(19) United States
(12) Patent Application Publication
(10) Pub. No.: US 2025/0243448 A1
(43) Pub. Date: Jul. 31, 2025

(54) BIOMIMETIC AEROSOL EXPOSURE SYSTEM (BAES) FOR CONCURRENT MULTI-SCALE MULTI-FACTOR IN-VITRO AIRWAY EXPOSURE STUDIES

(71) Applicants: Samantha Emma Sarles, Bel Air, MD (US); Edward C. Hensel, Rochester, NY (US); Risa J. Robinson, Greece, NY (US); Shehan Jayasekera, Rochester, NY (US); A. Gary DiFrancesco, Bristol, VA (US); Nathan C. Eddingsaas, Rochester, NY (US)

(72) Inventors: Samantha Emma Sarles, Bel Air, MD (US); Edward C. Hensel, Rochester, NY (US); Risa J. Robinson, Greece, NY (US); Shehan Jayasekera, Rochester, NY (US); A. Gary DiFrancesco, Bristol, VA (US); Nathan C. Eddingsaas, Rochester, NY (US)

(73) Assignee: Rochester Institute of Technology, Rochester, NY (US)

(21) Appl. No.: 19/035,644

(22) Filed: Jan. 23, 2025

Related U.S. Application Data
(60) Provisional application No. 63/624,119, filed on Jan. 23, 2024.

(51) Int. Cl. C12M 1/34; C12M 1/00; C12M 1/32

(52) U.S. Cl. CPC C12M 41/46; C12M 23/12; C12M 29/00

(57) ABSTRACT

Provided herein are novel in vitro biomimetic aerosol exposure systems designed to enhance the understanding of respiratory health impacts from aerosol inhalant devices, such as e-cigarettes. The systems and methods described herein improve biomimicry at multiple levels, including, system, macroscopic, and cellular levels, to provide more accurate and realistic exposure conditions. As described, these systems and methods address many limitations of existing smoking machines by offering better correlation between in vitro and in vivo conditions, improved particle deposition and dosimetry, and more realistic interactions between aerosols and biological samples. As various aspects, the biomimetic aerosol exposure system includes an emissions delivery system, an oral cavity module, a bifurcated exposure chamber, and a flow control subsystem.

### CROSS-REFERENCE TO RELATED APPLICATIONS

This application claims priority to U.S. Provisional Patent Application Ser. No. 63/624,119, filed on Jan. 23, 2024 and entitled “BIOMIMETIC AEROSOL EXPOSURE SYSTEM (BAES) FOR CONCURRENT MULTI-SCALE MULTI-FACTOR IN-VITRO AIRWAY EXPOSURE STUDIES”, the entirety of which is incorporated herein by reference.

### FIELD OF THE DISCLOSURE

The present disclosure relates generally to respiratory toxicology analyses and in vitro emissions testing systems, and more specifically to biomimetic aerosol exposure systems and methods of in vitro emissions testing that increase biomimicry of humans.

### BACKGROUND

Tobacco use remains the number one cause of preventable death in the United States, disproportionately affecting residents of rural areas, people who are financially disadvantaged, and adults who identify as gay, lesbian, or bisexual. The impact that electronic cigarettes will have on public health is not yet fully understood. For example, it remains unclear whether electronic cigarettes will have a net positive or negative effect on public health. In 2019, CDC reported that 27.5% of children under 18 identified as “current e-cigarette users”. The first cases of “electronic cigarette, or vaping, product use-associated lung injury” (EVALI) were reported to the US Center for Disease Control (CDC) in August 2019. Within five months, there were 2,561 reported hospitalized cases of EVALI including 55 deaths. As the COVID-19 pandemic unfolded, it was observed that inhaled nicotine use may increase the risk of SARS-CoV-2 diagnosis, compromise antiviral response, and lead to more detrimental clinical outcomes. A COVID-19 diagnosis is five times more likely in adolescents who ever used e-cigarettes and seven times more likely in youth who ever dual-used cigarettes and e-cigarettes.

Combinations of electronic cigarette products, human airway geometries, and user behaviors create barriers to understanding products' interaction with the human airway and their health effects. For example, the human airway is made up of a continuous flow path that constricts, turns, and branches into millions of small airway structures, and factors such as the characteristics of the aerosol inhalant device (such as electronic cigarettes), behavior of the user, and the anatomical structure of the human respiratory tract all affect what, how much, and where emissions from the inhaled aerosols deposit and interact with airway tissue. Thus, despite widespread publication of e-cigarette related work, many studies fail to directly link results to predicted health effects or regulatory outcomes, often due to non-repeatable results, lack of standardization, and unrealistic methods of exposure in the laboratory.

Moreover, current in vitro emissions systems (such as traditional cigarette smoking machines, programmable smoking and vaping machines, smoking robots, and vitrocell exposure systems), lack biomimicry, use unrealistic flow conditions, produce unrealistic aerosol dose, and provide inaccurate bio-mechanical cues to cell cultures, all of which limits researchers' ability to correlate in vitro outcomes with in vivo health effects. M any of these in vitro toxicology testing systems bear little resemblance to the geometric and flow conditions of the human airway. Other testing systems, sch as organ-on-a-chip systems, seek to achieve high fidelity local conditions but fail to capture a systems level view of either the airway or the flow conditions. Thus, current emissions systems do not have the ability to mimic both user behavior and geometric biomimicry for biological exposure.

### SUMMARY OF THE DISCLOSURE

In light of the foregoing, the present disclosure is directed to in vitro biomimetic aerosol exposure systems and methods of utilizing such systems, so as to enable respiratory health experts to better understand the causes and effects of aerosol inhalant device usage, including of e-cigarette associated lung injuries and other adverse health impacts associated with inhaled nicotine, cannabidiol (CBD), gases, solvents, chemicals, pathogens, and irritants. The biomimetic aerosol exposure systems and methods provide significant improvements over existing technologies in terms of airway particle deposition and dosimetry, improved correlation between in vitro and in vivo local environment conditions, and more realistic interactions between the flowing aerosol and biological samples. These and other advantages will become apparent based on the disclosure below.

According to one embodiment of the present disclosure, a biomimetic aerosol exposure system is provided. The biomimetic aerosol exposure system can include: an emissions delivery system device comprising an emissions outlet and configured to generate at least a first type of emissions; an oral cavity module comprising a biomimetic exposure chamber, an inlet, and an outlet, wherein the first exposure chamber mimics one or more parameters of a human oral cavity; an emissions delivery system adapter configured to connect at least a portion of the emissions delivery system device including the emissions outlet at a first interface with the inlet of the oral cavity module at a second interface; and a bifurcated exposure chamber operatively connected to the outlet of the oral cavity module.

In an aspect, the emissions delivery system adapter, the oral cavity module, and the bifurcated exposure chamber create a flow path that mimics the biology of human respiration.

In an aspect, the system can further include a flow control subsystem configured to control a flow of clean air and a flow of emissions from the emissions delivery system device throughout the flow path.

In an aspect, the emissions delivery system adapter comprises an intermediate region between the first interface where the emissions delivery system device is received and the second interface where the oral cavity module inlet is received, and the intermediate region provides a streamlined flow path configured to minimize mass loss of the emissions.

In an aspect, the emissions delivery system adapter is formed from silicone rubber.

In an aspect, the oral cavity module further comprises one or more alternate inlets, at least one of which is located approximately at a region corresponding to a soft palate of a human.

In an aspect, the biomimetic exposure chamber of the oral cavity module has an open volume of from about 24 cm³ to about 75 cm³, a surface area of from about 75 cm² to about 230 cm², and/or a L_{COM} of from about 6 cm to about 20 cm.

In an aspect, the bifurcated exposure chamber comprises an inlet, a first and a second outlet, and an enclosure having a lid defining an interior volume of the enclosure.

In an aspect, the bifurcated exposure chamber further comprises a bifurcated flow path part defining a bifurcated flow channel, a multiple well plate, and one or more cell culture inserts disposed within the interior volume of the enclosure.

In an aspect, the bifurcated flow path part is configured to lay on top of the multiple well plate within the enclosure and to guide at least the first type of emissions from the emissions delivery system device and through the bifurcated flow channel.

According to another embodiment of the present disclosure, a bifurcated exposure chamber for use in a biomimetic aerosol exposure system is provided. The bifurcated exposure chamber can include: an enclosure defining a first end, a second end, and an interior volume; an inlet disposed at the first end and configured to be coupled to an inlet tubing; and a first and a second outlet disposed at the second end and configured to be coupled to exit tubing;

In an aspect, the first and second ends are arranged 180° from one another.

In an aspect, the bifurcated exposure chamber can further include: a bifurcated flow path part defining a bifurcated flow channel through the bifurcated flow path part, wherein the bifurcated flow path part is disposed or configured to be disposed within the interior volume of the enclosure.

In an aspect, the bifurcated flow channel comprises a first channel in communication with the inlet and bifurcates into a second and a third channel in communication with the first and second outlets, respectively.

In an aspect, an overall length of the bifurcated flow path through the bifurcated flow path part is from about 63 mm to about 200 mm, a length of the first channel before the bifurcation is from about 26 mm to about 80 mm, a width of the first channel before the bifurcation is from about 8 mm to about 25 mm, a height of the first channel before the bifurcation is from about 8 mm to about 25 mm, a length of each of the second and third channels is from about 36 mm to about 110 mm, a width of each of the second and third channels is from about 7 mm to about 22 mm, and/or a height of each of the second and third channels is from about 4 mm to about 15 mm.

In an aspect, the bifurcated flow path has a branching angle of from about 30° to about 60°.

In an aspect, the bifurcated flow path part comprises through-holes configured to receive cell culture inserts when assembled together with a multi-plate well within the interior volume of the enclosure.

In an aspect, the bifurcated exposure chamber can further include: the multi-plate well; and one or more cell culture inserts having one or more cell culture exposure surfaces; wherein the bifurcated flow path part is configured to lay on top of the multi-well plate within the enclosure and to guide aerosol flow tangentially across the one or more cell culture exposure surfaces within the bifurcated exposure chamber using physiologically relevant geometry and flow conditions.

According to another embodiment of the present disclosure, a method for mimicking respiration behavior is provided. The method can include providing a flow path through a biomimetic aerosol exposure system, wherein the biomimetic aerosol exposure system comprises: an emissions delivery system device comprising an emissions outlet and configured to generate at least a first type of emissions; an oral cavity module comprising a biomimetic exposure chamber, an inlet, and an outlet, wherein the first exposure chamber mimics one or more parameters of a human oral cavity; an emissions delivery system adapter configured to connect at least a portion of the emissions delivery system device including the emissions outlet at a first interface with the inlet of the oral cavity module at a second interface; and a bifurcated exposure chamber operatively connected to the outlet of the oral cavity module.

In an aspect, the method can further include: initiating a flow control subsystem of the biomimetic aerosol exposure system; and supplying at least the first type of emissions from the emissions delivery system device to the flow path through the biomimetic aerosol exposure system.

These and other aspects of the various embodiments will be apparent from and elucidated with reference to the embodiments described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a block diagram schematic of a biomimetic aerosol exposure system illustrated in accordance with aspects of the present disclosure.

FIG. 2 is a cross section view of an emissions device adapter illustrated in accordance with aspects of the present disclosure.

FIG. 3 is an illustration of calculated wall profiles for use in manufacturing an emissions device adapter illustrated in accordance with aspects of the present disclosure.

FIG. 4 is a side-by-side comparison of an oral cavity of a human with an oral cavity module illustrated in accordance with aspects of the present disclosure.

FIG. 5 is an enlarged cross section view of the emissions device and the oral cavity module of a biomimetic aerosol exposure system illustrated in accordance with aspects of the present disclosure.

FIG. 6A is an enlarged cross section view of an inlet portion of the oral cavity module illustrated in accordance with aspects of the present disclosure.

FIG. 6B is an enlarged cross section view of a human oral cavity mimetic exposure chamber of the oral cavity module illustrated in accordance with aspects of the present disclosure.

FIG. 6C is a downstream portion of the oral cavity module illustrated in accordance with aspects of the present disclosure.

FIG. 6D is an outlet of the oral cavity module illustrated in accordance with aspects of the present disclosure.

FIG. 7 is a series of side-by-side views of the flow volume within the oral cavity module illustrated in accordance with aspects of the present disclosure.

FIG. 8 is an exploded view of a bifurcated exposure chamber illustrated in accordance with aspects of the present disclosure.

FIG. 9 is a photograph of a bifurcated exposure chamber and certain associated components shown in accordance with aspects of the present disclosure.

FIG. 10 is a diagram of a cell culture insert for use in a bifurcation exposure chamber illustrated in accordance with aspects of the present disclosure.

FIG. 11 is an illustration showing additional configurations of the biomimetic aerosol exposure system in accordance with aspects of the present disclosure.

FIG. 12 is a schematic of another biomimetic aerosol exposure system illustrated in accordance with further aspects of the present disclosure.

FIG. 13A is a schematic of another biomimetic aerosol exposure system illustrated in accordance with still further aspects of the present disclosure.

FIG. 13B is a schematic of another biomimetic aerosol exposure system illustrated in accordance with yet further aspects of the present disclosure.

FIG. 14 is a photograph of a biomimetic aerosol exposure system assembled and shown in accordance with aspects of the present disclosure.

FIG. 15 is a flowchart illustrating a method of mimicking human respiratory behavior in accordance with aspects of the present disclosure.

### DETAILED DESCRIPTION OF THE EMBODIMENTS

As mentioned above, the present disclosure is directed to in vitro biomimetic aerosol exposure systems and methods of utilizing such systems. These biomimetic aerosol exposure systems and methods enable respiratory health experts to better understand the causes and effects of aerosol inhalant device usage, including of e-cigarette associated lung injuries and other adverse health impacts associated with inhaled nicotine, cannabidiol (CBD), gases, solvents, chemicals, pathogens, and irritants. The biomimetic aerosol exposure systems and methods provide significant improvements over existing technologies in terms of airway particle deposition and dosimetry, improved correlation between in vitro and in vivo local environment conditions, and more realistic interactions between the flowing aerosol and biological samples.

In accordance with particular aspects, the present disclosure appreciates a number of technical challenges evident in conventional aerosol exposure systems: (1) aerosol particle deposition, and hence dose, in aerosol exposure systems depend upon the flow path geometry (including under both transient and steady state system flow conditions), and therefore the deposition of aerosol along the respiratory tract (between the mouth and the location of study) obscures the relationship between in vivo and in vitro dosimetry; (2) aerosol particle deposition and local flow conditions in machine puffing systems depend upon puff and respiratory topography, referred to as the “system flow conditions,” and therefore differences between the in vivo inhalation and in vitro machine puffing conditions limit the extrapolation of laboratory findings to human respiratory exposure and cell response, and further presents issues related to local flow conditions experienced by cell cultures like wall shear stress; (3) while aerosol particle deposition naturally occurs in machine-puffing systems, differences between in vivo and in vitro local cell environments limit the extrapolation of laboratory findings to human respiratory exposure and cell response; (4) the viability of cells can vary under no flow conditions, transverse flow of clean air, and transverse flow of ENDS aerosol, and current exposure systems do not provide realistic dose or mechanical cues to cell cultures during aerosol exposure; and (5) acute exposure of cell cultures under high fidelity dose, geometry, and flow conditions are not possible in traditional machine puffing emissions systems, and therefore these systems do not provide the most accurate risk assessments of inhaled aerosol products in physiologically-relevant environments.

Furthermore, regarding the behavioral aspects associated with aerosol inhalant devices, it is appreciated that characteristics such as aerosol outlet hydraulic diameter, power, and e-liquid pH of such aerosol inhalant devices vary significantly. These product attributes can change puffing topography, including flowrate and puff duration, which can have an effect on emissions and should be considered in both analytical and biological testing of inhaled aerosol emissions. Although it is documents that use topography effects emissions and that users puff at variable flow rates within a session, most existing emissions systems are not capable of variable flow rates or simulating inhales between puffs or exhales.

Additionally, there are significant limitations when comparing human health response to in vivo and in vitro studies of health effects. Biological studies related to tobacco use, for example, are generally limited to non-invasive, low risk observation. Saliva and oral swabs can be used as bio samples for human subjects research since they are easily accessible and because oral health gives insight into systemic health. The oral cavity immune system consists of resident immune cells in the salivary glands, immune cells that migrate from systemic circulation, and the mucosal immune system. The immune system of the oral cavity is in bidirectional communication with the gut and systemic immune systems. However, the current state of knowledge indicates that levels of at least some biomarkers in salvia do not correlate well with levels of in systemic circulation and therefore may not be a good measure of systemic health. Further, salivary biomarkers such as pro-inflammatory cytokines and immunoglobulins are associated with chronic and acute stress and may confound results. Nevertheless, the use of animal models is not an ideal alternative for mechanistic studies involving the human airway, because most small animals are nose breathers and the makeup and structure of airway epithelium differs significantly between humans and other species such as mice.

Despite advancements that have made in aerosol exposure systems, many studies still use unrealistic exposure techniques, arbitrary doses, and non-relevant cell lines. Mechanical cues have also been shown to affect cellular behavior (e.g., fluid shear has been shown to alter protein expression), but existing aerosol exposure systems generally do not account for effects different mechanical cues on the biological response to aerosolize compounds.

Thus, in accordance with various aspects, the present disclosure increases biomimicry at three levels: (1) the systems level with the ability to perform puffing and ambient air inhalation between puffs, mimicking how a human uses, for example, an electronic cigarette; (2) the macroscopic level where the flow path to emissions/characterization and biological exposure subsystems utilize geometries that mimic the human airway, including surface topographies, turns, and a bifurcation; and (3) the cellular level where the free stream angle of aerosol induces fluid shear on cells, mimicking physiological conditions. As described herein, the biomimetic aerosol exposure systems and methods of the present disclosure improve upon conventional emissions systems to generate physiologically relevant in vitro models of exposure to inhaled aerosols, but not limited to including tobacco and/or nicotine.

### First Embodiment

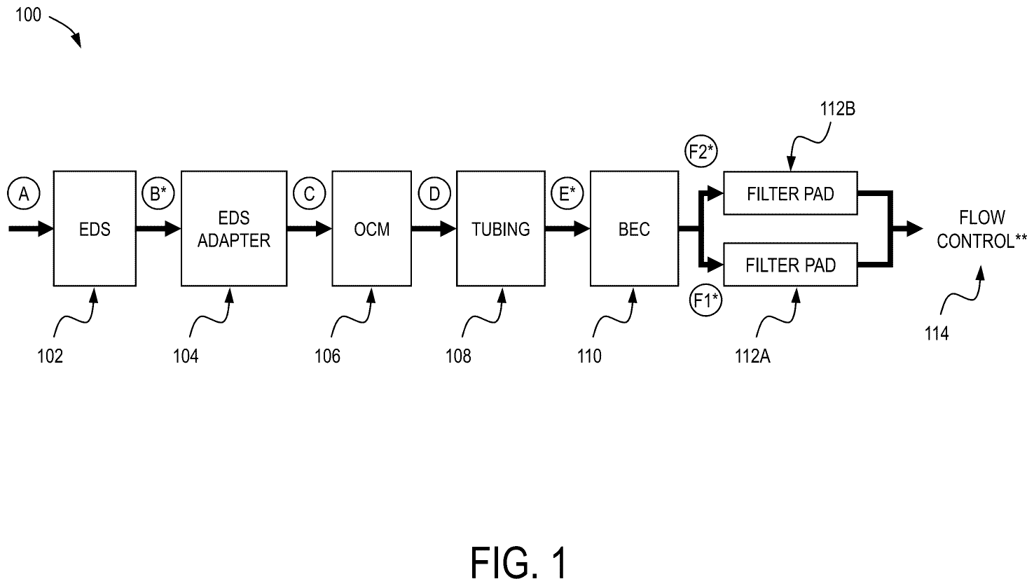

Turning now to FIG. 1, a schematic of a biomimetic aerosol exposure system 100 is illustrated in accordance with aspects of the present disclosure. As shown, the biomimetic aerosol exposure system 100 may include: an emissions delivery system 102, sometimes referred to as an EDS; an EDS adapter 104; an oral cavity module 106, sometimes referred to as an OCM; a tubing portion 108; a bifurcated exposure chamber 110, sometimes referred to as a BEC; one or more filter pads 112A, 112B; and a flow control subsystem 114. Each of these components are described in more detail below.

### Emissions Delivery System (EDS)/Device Under Testing (DUT)

According to certain aspects of the present disclosure, the biomimetic aerosol exposure system 100 can include an EDS device that is used to introduce the aerosol emissions. In embodiments, the EDS device may be a nicotine delivery device, such as an electronic cigarette. In such cases, the EDS may be referred to as an electronic nicotine delivery system or ENDS device. However, it is contemplated that similar devices may be used to delivery other aerosolized compounds, including but not limited to cannabidiol (CBD), tetrahydrocannabinol (THC), and other gases, solvents, chemicals, pathogens, and/or irritants. In more general embodiments, such devices used to deliver non-nicotine emissions may be referred to simply as a device under testing, or DUT.

Notably, it is appreciated herein that the mount and connection of the EDS device 102 to the rest of the biomimetic aerosol exposure system 100 is an important consideration. For example, if an electronic cigarette is puffed horizontally or with the mouthpiece lower than the distal end, it increases the risk of “dry puffing”. This phenomenon occurs when the wick of an e-cigarette's atomizer is not bathed in liquid and becomes dry. When power is delivered to the coil, the wick is burned instead of e-liquid being aerosolized. Accordingly, the EDS device 102 may be mounted and/or supported at a particular angle below horizontal, such as at least about 30° below horizontal.

### EDS/DUT Adapter

Next, according to certain aspects of the present disclosure, the biomimetic aerosol exposure system 100 can include an EDS or DUT adapter 104, which is used to connect the EDS device 102 to the rest of the biomimetic aerosol exposure system 100. In particular embodiments, the EDS adapter 104 provides an airtight seal at the connection between the DUT device 102 and the emissions system 100. Further, because the DUT device 102 may be repeatedly connected and disconnected during an experiment, the airtight seal may be releasable and repeatable through the use of a customized gasket.

As described herein, it is also appreciated that mass loss between the EDS device 102 (e.g., an e-cigarette) and biological exposure or emissions capture is a significant barrier in quantification of biological response to emissions. Conventional adapters may induce deposition within the flow path or the system before the aerosol ever arrives at the exposure chamber or the capture system, potentially under-reporting fundamental emissions outcomes. As such, the EDS adapters 104 of the present disclosure are designed to minimize abrupt changes in cross-sectional area of the flow path and reduce the cumulative flow path volume between the exit plane of the DUT and emissions system entrance. In embodiments, the EDS adapter 104 may be customized to the particular geometries of the EDS device 102, especially the flow outlet of the EDS device 102.

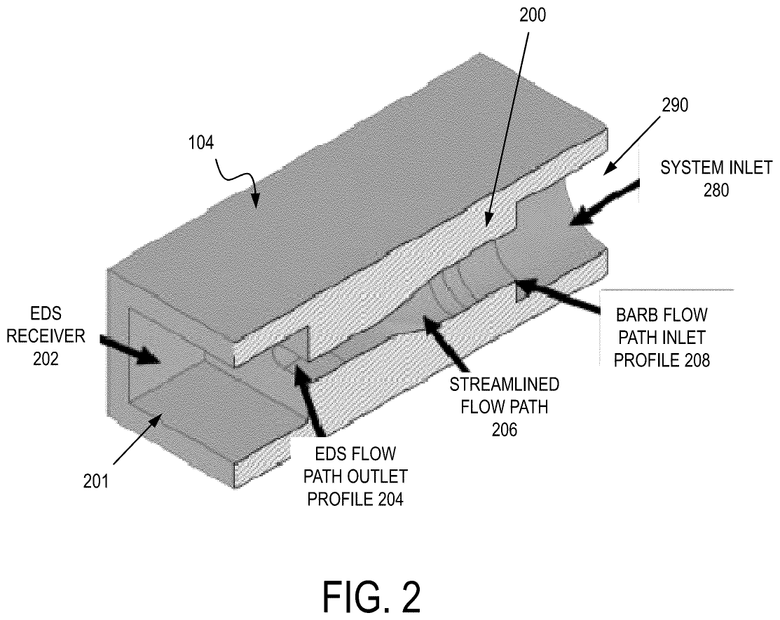

For example, with reference to FIG. 2, an orthographic cross-section view of a modeled EDS adapter 104 is illustrated in accordance with aspects of the present disclosure. As shown in the example of FIG. 2, the EDS adapter 104 comprises an adapter body 200 having a multi-section, continuous interior cavity extending from a first end 201 where the EDS device 102 is received to a second end 290 where a system inlet 280 is received. In embodiments, the adapter body 200 can include the following sections: (1) a first interface 202 where the EDS device is received (i.e., an EDS receiver portion); (2) a second section 204 providing an EDS flow path outlet profile; (3) a third section 206 providing streamlined flow path; (4) a fourth section 208 providing a barb flow path inlet profile; and (5) a second interface 280 providing an system inlet receiving portion.

In particular embodiments, the transition in the flow path diameter between the EDS device exit located at the EDS flow path outlet profile 204 and emissions system inlet 280 is configured to minimize mass loss of the emissions. For example, a sigmoid function can be used to identify the radii of coincident ellipses, whose tangent functions exhibit C¹ continuity. The adapter body spline connects the outlet of the EDS device 102 to the inlet of a coupling on the filter pad holder. In embodiments, the radii are calculated as:

$$f = \frac{1}{1 + {e}^{- z}}$$

$$where z = G π ( \frac{x - 0.5}{L} ) , G = \frac{x}{L} ,$$

L=transition path distance, and x=plane distance.

Points for line segments of horizontal and vertical ellipse radii can be determined according to:

$${P}_{x} = {x}_{0} ( 1 - f ) + f {x}_{L}$$

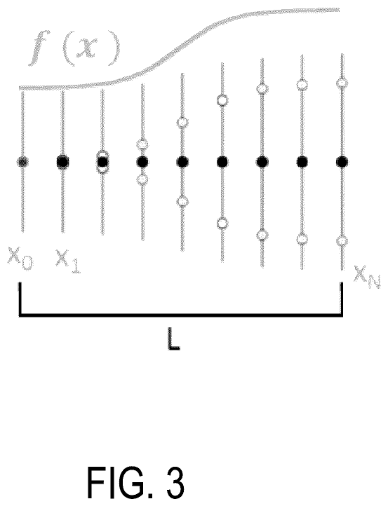

For example, in certain embodiments, computer aided design software can be utilized to create planes at x₀ and x_{L} and segmented with planes every 10% of L. Ellipses with radii P_{y} and Pₓ, as calculated above, are placed on each plane. Exemplary profiles the define the flow path transition of the streamlined adapter section 206 is shown in FIG. 3.

Using such profiles, a cut can be lofted through a solid block of material to create a continuous flow path 206 within the adapter body 200. The receiving sections 201, 280 can then be modeled based on different mechanical connectors and EDS devices 102. A mold can be fabricated and used to mold the final adapter 104. For example,

In embodiments, the adapter body 200 may be molded from silicone rubber, however, other materials and combinations of materials may be suitable.

### Oral Cavity Module (OCM)

Next, according to certain aspects of the present disclosure, the biomimetic aerosol exposure system 100 can include an oral cavity module 106, which provides a human oral cavity mimetic exposure chamber appropriate for conducting aerosol deposition, cell, exposure, and toxicity studies of relevant cell lines and/or tissues. As a result, the oral cavity module 106 provides enhanced biomimicry in comparison to traditional smoking machines and exposure systems, especially in terms of oral cavity particle deposition and dosimetry, and improved correlation between in vitro, in silico, and in vivo conditions.

H ere, it is appreciated that the oral cavity is an important part of the human airway and a primary defense mechanism for the body. However, currently available emissions systems are not equipped for investigations into inhaled environments impact on oral health. The transit length to exposure chambers of commercially available exposure systems is not representative of transit to the oral cavity and therefore dosimetry and particle deposition is not representative of in vivo exposure. Much current literature exposes bronchial or lung epithelium to a dose that is indefinite and often not repeatable because of aerosol loss in transit. Results that quantify cytotoxicity, genotoxicity, inflammatory response, and oxidative stress are not representative of the entire airway. N either mass loss nor biological response in the oral cavity is well-understood, and current emissions systems do not provide the geometry to simulate deposition in the oral cavity.

Furthermore, respiration of clean air is an aspect of human behavior that is often neglected in conventional machine puffing systems. For example, the Independent Holistic Air-Liquid aerosol exposure system (InHALES) developed by Steiner et al. is the first emissions system with the ability to simulate regular breathing between puffing. However, aerosol delivery was validated inside of the pumps but not in the entire airway model, where many cell cultures are exposed to test environments. Also, mass delivery (ng/cm²) inside of the primary pump was not repeatable. Further, cells exposed to lab air flowing through the apparatus caused a significant difference in cell viability compared to an incubator control when a “deep inhale” (an inhale to the lungs) was simulated. And command flow was not achieved by the InHALES when the resistance of a cigarette was added to the inlet.

It is also appreciated herein that downstream from the oral cavity, an inhale moves through the pharynx, the larynx, and into the tracheobronchial region. In humans, the tracheobronchial region dominates particle deposition, protecting the fragile downstream airway, yet geometries that mimic this have not been used or validated in analytical emissions work.

Accordingly, as described herein, the oral cavity module 106 can be configured to integrate a human oral cavity mimetic exposure chamber, integrate an oropharynx model to control flow paths from the oral cavity and sinus cavity into the pharynx, and provide the ability to simulate inhalation of clean air between emissions thereby enabling simulation of human puffing behavior with the exposure system 100.

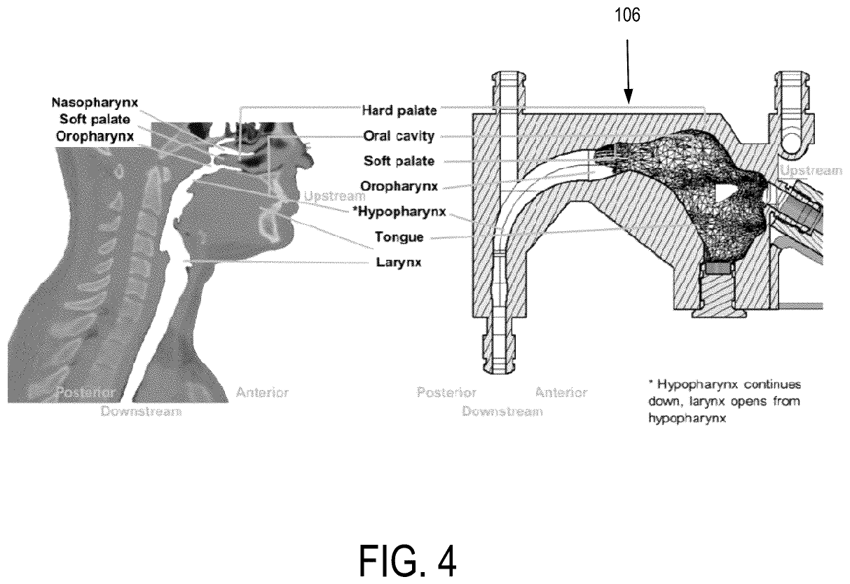

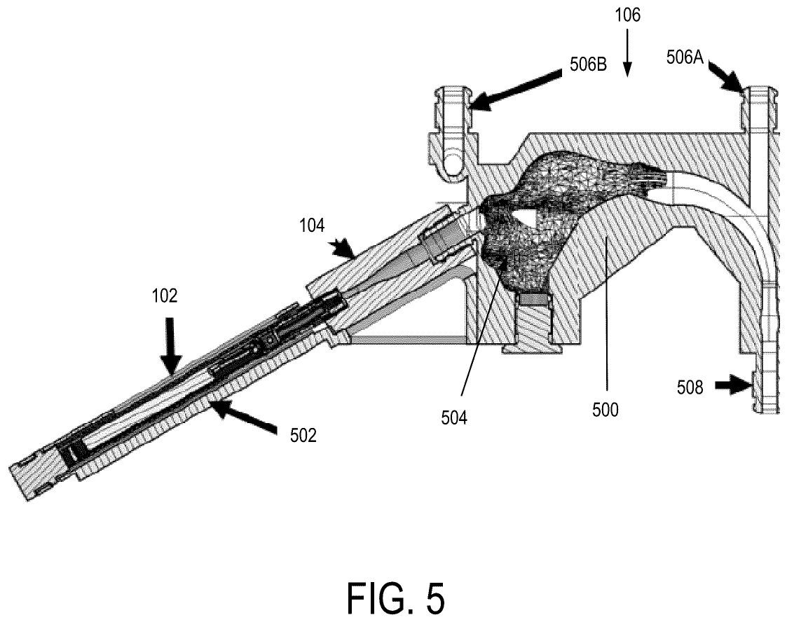

With reference to FIG. 4, a comparison of the geometry of an oral cavity module with a representative human anatomy is shown. With reference to FIG. 5, the application of the oral cavity module 106 within a biomimetic aerosol exposure system 100 is illustrated in accordance with various aspects of the present disclosure. For example, as shown in FIG. 5, the EDS device 102 supported at a desirable angle of inclination by a positioning jig 502 and is attached to the oral cavity module 106 via the ED S adapter 104. The oral cavity module 106 comprises a module body 500 defining several interior regions including at least a biomimetic exposure chamber 504, one or more alternate inlets 506A, 506B to allow modularity with the ability to connect alternative pumps to the oral cavity module 106, and an outlet 508 leading downstream. In particular embodiments, at least one of the alternate inlets 506A may be located approximately at a region corresponding to the soft palate and can be utilized to mimic the inlet from the nasopharynx to the oropharynx.

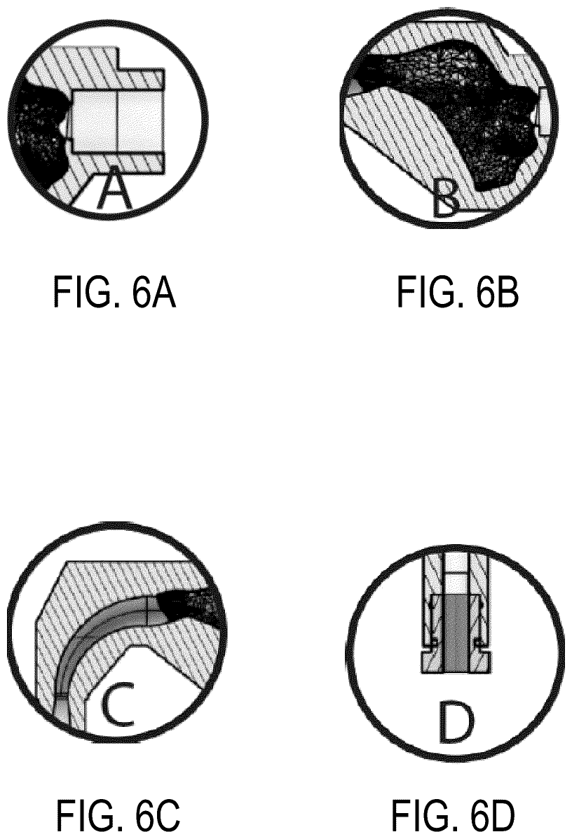

With reference to FIGS. 6A-6D, further details of the oral cavity module 106 are illustrated in accordance with aspects of the present disclosure. For example, as shown in FIG. 6A, the oral cavity module 106 can include an inlet 600 configured to mimic human lips and the entryway to the larynx. In some embodiments, a mechanical connector compatible with the outlet of the EDS adapter 104 can be modeled onto the “lips” of the inlet 600. In embodiments, the mechanical connector can be rotated at a desirable angle of inclination (e.g., about 30°) from horizontal to reflect the angle that a user might hold an e-cigarette in their mouth, for example. As shown in FIG. 6B, the biomimetic exposure chamber 504 can have a geometry that is modeled based on actual human oral cavity dimensions, and/or based on a statistical representation thereof. As shown in FIG. 6C, the oral cavity module 106 can include a spline between the regions analogous to the oropharynx and the hypopharynx. Finally, as shown in FIG. 6D, the oral cavity module 106 can include an adapter located downstream and configured to connect to the bifurcated exposure chamber 110.

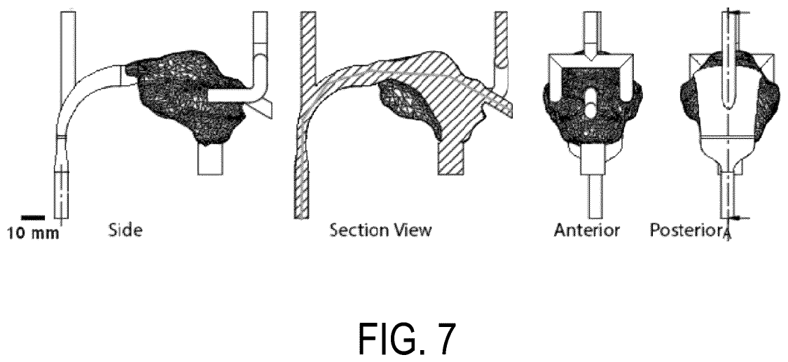

With reference to FIG. 7, a schematic of the fluid space of the oral cavity module 106 is illustrated from different perspectives in accordance with various aspects of the present disclosure. In particular embodiments, key anatomical dimensions of the oral cavity module 106 can include volume (V), surface area (SA), and length of the oral cavity module 106 (L_{OCM}). For example, in embodiments, the volume can be from about 24 cm³ to about 75 cm³, the surface area can be from about 75 cm² to about 230 cm², and the L_{OCM} can be from about 6 cm to about 20 cm. In specific embodiments, the volume can be about 49.62 cm³, the surface area can be about 152.98 cm², and the L_{OCM} can be about 13.20 cm. However, other values and ranges are possible depending on various factors, such as the age of the modeled cohort.

### Tubing

Next, according to certain aspects of the present disclosure, the biomimetic aerosol exposure system 100 can include tubing 108 which connects the outlet 508 of the oral cavity module 106 with a bifurcated exposure chamber 110 having an internal flow path modeling the first lung bifurcation. In embodiments, the tubing 108 may be flexible tubing formed from, for example, polyethylene. In embodiments, the tubing 108 may have a length of from about 14 cm to about 45 cm, including about 28.4 cm. However, other values and ranges are possible depending on various factors, such as the age of the modeled cohort.

### Bifurcated Exposure Chamber (BEC)

Next, according to certain aspects of the present disclosure, the biomimetic aerosol exposure system 100 can include a bifurcated exposure chamber 110 that is configured to create a traverse flow path over a multiple well plate and be integrated into the biomimetic aerosol exposure system 100 downstream from the oral cavity module 106. As described herein, the bifurcated exposure chamber 110 mimics the fluid mechanics and geometry of at least the first lung bifurcation, creating a biomimetic instrument in which analytical studies and biological exposures can take place using identical geometry.

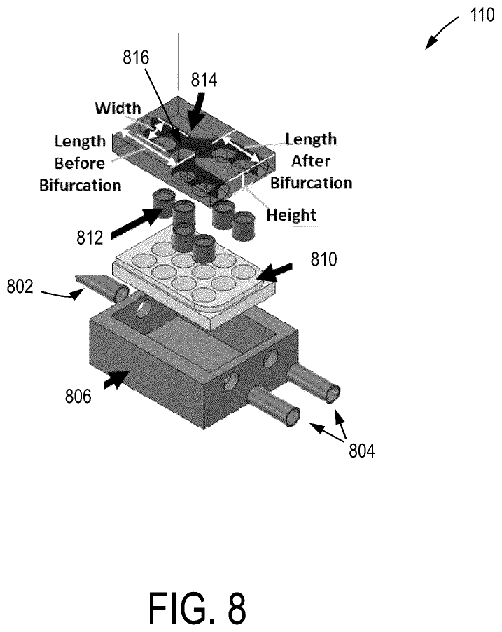

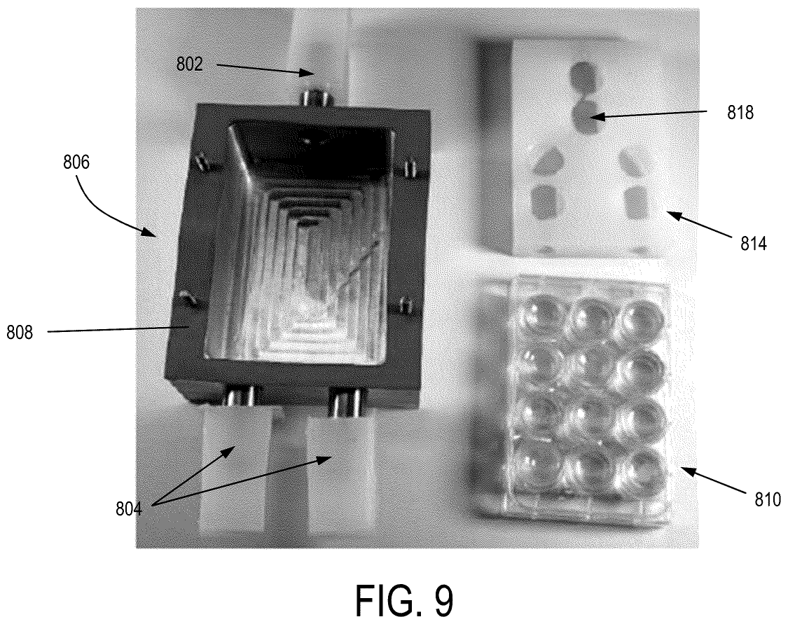

With reference to FIGS. 8 and 9, an exemplary bifurcated exposure chamber 110 assembly is illustrated in accordance with various aspects of the present disclosure. As shown in FIG. 8, an exploded view of a schematic illustrating the bifurcated exposure chamber 110 is illustrated, whereas FIG. 9 shows a photograph of certain components of the bifurcated exposure chamber 110.

As shown in these figures, the bifurcated exposure chamber 110 can include inlet tubing 802 and outlet tubing 804, an enclosure 806, a lid 808 with a rubber gasket (not shown), and a multiple well plate 810, cell culture inserts 812, and a bifurcated flow path part 814.

In embodiments, the enclosure 806 and lid 808 may be milled from aluminum, and the lid 808 may include a gasket made of silicone having a hardness 60D. The tubing 802, 804 may be steel tubing, can have a length of about 50.8 mm, and may be sealed to the enclosure 806 via a sealant (e.g., Hylomar Universal Blue Sealant).

As described herein, the bifurcated flow path part 814 is configured to lay on top of the multi-well plate 810 within the enclosure 806 and to guide aerosol flow tangentially across the cell culture exposure surfaces within the bifurcated exposure chamber 110 using physiologically relevant geometry and flow conditions. In embodiments, the bifurcated exposure chamber 110 can be configured to accept commercially available multiple well plates.

A bifurcated flow channel 816 extends through the bifurcated flow path part 814 allowing the aerosol flow across the cell multi-well plate 810. The bifurcated flow channel 816 may include a single channel extending from the inlet tubing 802, which then bifurcates into two channels connecting to the outlet tubing 804. The bifurcated flow channels 816 can have a cross section such that the flow path of each generation is aligned along the centerline of the wells of the multi-well plate 810. In some embodiments, the bifurcated flow channels 816 can have a rectangular cross section.

The height of the bifurcated flow path part 814 produces interference that acts together with the gasket (not shown) to ensure that the chamber 110 is airtight during operation. As described herein, the bifurcated flow path part 814 can be configured to model a symmetrical lung with a rectilinear cross-section and branching angles that may be customized as desired. In particular embodiments, geometrically relevant dimensions can be implemented using an idealized model of asymmetric lung with branching angles as described in E. R. Weibel, et al., Morphometry of the human lung, vol. 1: Springer, 1963. These geometries may define a width, length, and/or height before bifurcation as well as a different width, length, and/or height after bifurcation, as shown in FIG. 8.

For example, in specific embodiments, the overall length of the bifurcated flow path through the bifurcated flow path part 814 can be from about 63 mm to about 200 mm, including about 53.50±0.01 mm. The length of the channel before the bifurcation can be from about 26 mm to about 80 mm, including about 53.50±0.01 mm. The width of the channel before the bifurcation can be from about 8 mm to about 25 mm, including about 16.45±0.01 mm, and the height of the channel before the bifurcation can be from about 8 mm to about 25 mm, including about 16.64±0.01 mm. Downstream from the bifurcation, the length of each of the channels can be from about 36 mm to about 110 mm, including about 73.69±0.01 mm, the width of each of the channels can be from about 7 mm to about 22 mm, including about 14.32±0.01 mm, and the height of each of the channels can be from about 4 mm to about 15 mm, including about 9.97±0.01 mm.

In embodiments, the branching angle of the bifurcated flow path can be from about 30° to about 60°, including about 48°, from the centerline of the bifurcation. In further embodiments, the bifurcated flow path part 814 can be formed from a polymer material, such as silicone rubber and/or polydimethylsiloxane (PMDS).

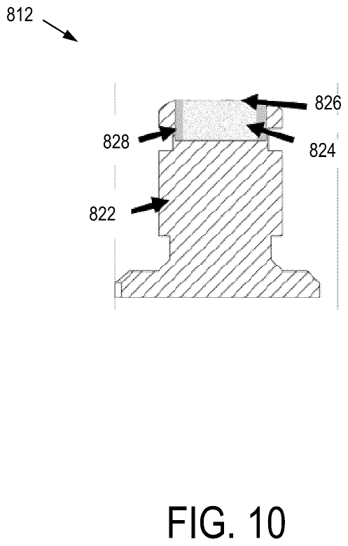

Through-holes 818 (shown in FIG. 9) within the bifurcated flow path part 814 can be configured in size and shape to accept the cell culture inserts 812 when assembled together with the multi-plate well 810. As described herein, the cell culture inserts 812 can provide a culture system for monolayers at an air-liquid interface. For example, with reference to FIG. 10, a schematic of a cell culture insert 812 comprising an insert body 822, a scaffold 824 for supporting the cell monolayer 826 and surrounded by culture media 828. In embodiments, the cell culture insert 812 can have a height of from about 9 mm to about 20 mm, including about 19.05 mm, can have an outer diameter of from about 10 mm to about 30 mm, including about 20.93 mm (to fit into a standard 12-well plate). The portion of the insert 812 that supports and holds the cell culture (i.e., the scaffold 824, the cell monolayer 826, and the culture media 828) can have a volume of from about 0.5 mL to about 1.5 mL, including about 0.97 mL, with a depth of from about 2 mm to about 5.7 mm, including about 3.81 mm, and a diameter of from about 9 mm to about 27 mm, including about 18.03 mm.

Accordingly, as described herein, the cell culture inserts 812 are configured to measure mass deposition or to culture cells in each well of the multi-well plate 810, which would be exposed to the open channels beneath the bifurcated flow path part 814. In embodiments, these components are modular such that they can be attached in a variety of configurations with analytical instruments inserted into the flow path at several locations.

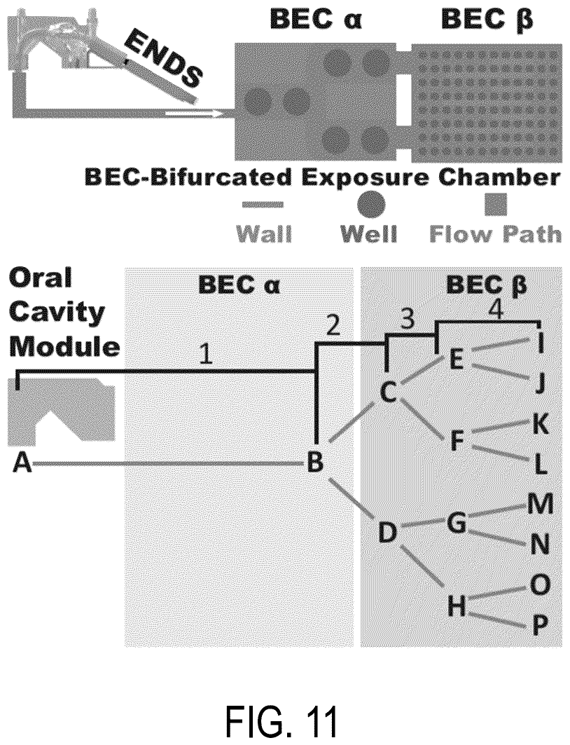

Turning to FIG. 11, it should also be appreciated that multiple bifurcated exposure chambers 110 can be linked in series and/or in parallel to mimic different generations of branching within the lungs. The multi-well plates 810, the flow path part 814, and the inserts 812 can be varied along the flow path as desired. Similarly, cell cultures can be placed as desired along these flow paths. The table below provides certain parameters associated with each generation of connected bifurcated exposure chamber 110 in accordance with certain aspects of the present disclosure:

TABLE 1
Dimensions of flow channels by lung generation
in a biomimetic aerosol exposure system

| | | Minimum | | | |
|---|---|---|---|---|---|
| | Number | Target Wall | Maximum Wall | Given | Given |
| | of | Shear Stress | Target Shear | Channel | Channel |
| Generation | Channels | (fₘᵢₙ) | Stress (fₘₐₓ) | Width (w) | Height (h) |
| [—] | [—] | [Pa] | [Pa] | [m] | [m] |
| 1 | 1 | 0.0025 | 0.02 | 0.0166 | 0.0166 |
| 2 | 2 | 0.02 | 0.03 | 0.0150 | 0.0100 |
| 3 | 4 | 0.03 | 0.05 | 0.0081 | 0.0075 |
| 4 | 8 | 0.03 | 0.05 | 0.0075 | 0.0050 |

### Filter Pads

Next, according to certain aspects of the present disclosure, the biomimetic aerosol exposure system 100 can include one or more filter pads 112A, 112B downstream from the bifurcated exposure chamber 110 (or downstream from multiple bifurcated exposure chambers 110, as the case may be). Each filter pad 112A, 112B may be connected to the flow path downstream from the outlets 804 of a bifurcated exposure chamber 110, and can be configured to capture testing emissions. In embodiments, the filter pads 112A, 112B may be contained within one or more filter pad holders (not shown), which may be part of an emissions capture subsystem. As described herein, system flow rate can be measured downstream from the filter pads 112A, 112B with a system flow meter and a calibrated orifice plate. The filter pads 112A, 112B may be removeable such that the mass of each of the filter pads 112A, 112B can be measured before, after, and/or in-between experiments, as well as removed and replaced.

### Flow Control Subsystem

Next, according to certain aspects of the present disclosure, the biomimetic aerosol exposure system 100 can include a flow control subsystem 114, which may be connected downstream to the filter pads 112A, 112B via the filter pad holders (not shown). As described in more detail below, the flow control subsystem can include a variety of components for measuring and controlling the flow rates at each of the locations along the biomimetic aerosol exposure system 100.

### Oral Clean Air Inhalation Subsystem

According to certain embodiments, the biomimetic aerosol exposure system 100 can also include an oral clean air inhalation subsystem, which is configured to insert the EDS device 102 into the EDS adapter 104 for an airtight connection to the OC M 106 during puffing and retracts the EDS device 102 at the conclusion of a puff for clean air inhalation through the same system inlet. In some embodiments, an L12-R micro linear servo (Actuonix Motion Devices, British Columbia, Canada) can be mounted co-axially aligned with the OCM inlet. A connector that holds an EDS device 102 onto the arm of the actuator can be designed.

### Second Embodiment

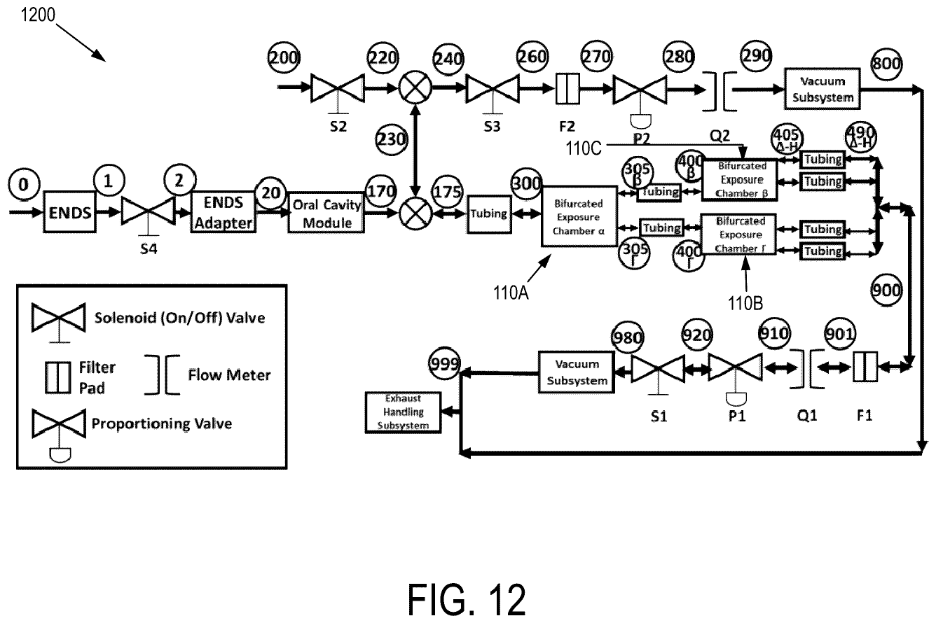

Turning now to FIG. 12, a more comprehensive system architecture for a biomimetic aerosol exposure system 1200 is illustrated in accordance with further aspects of the present disclosure. Here, the biomimetic aerosol exposure system 1200 can include each of the components described above, including an emissions delivery system 102, sometimes referred to as an EDS; an EDS adapter 104; an oral cavity module 106, sometimes referred to as an OCM; a tubing portion 108; a bifurcated exposure chamber 110, sometimes referred to as a BEC; one or more filter pads 112A, 112B; and a flow control subsystem 114. As shown in FIG. 12, the biomimetic aerosol exposure system 1200 is adapted for a nicotine experiment, thus the EDS 102 is labeled ENDS (i.e., electronic nicotine delivery system), etc. A s further shown, the system 1200 includes a first generation bifurcated exposure chamber 110A and two second generation bifurcated exposure chambers 110B, 110C.

The flow control subsystem 114 is also shown in more detail, which can include a number of solenoid valves, filter pads, flow meters, proportioning valves, a first vacuum subsystem operatively connected downstream from the bifurcated exposure chambers 110A, 110B, 110C, a second vacuum subsystem operatively connected upstream from the bifurcated exposure chambers 110A, 110B, 110C, and an exhaust handling subsystem operatively connected downstream from the vacuum subsystems.

As shown in FIG. 12, state numbers are shown inside of circles and given throughout the system to identify key system locations. In embodiments, room air enters the system at a primary inlet, State 0, or a secondary inlet, State 200. The “ENDS” is the DUT and aerosol generator. States 200-290 are the nasal cavity subsystem. A ‘T’ shaped pipe can extend upward from the oropharynx flow path with one side of the pipe an inlet for ambient air (state 200) through a solenoid valve (S2). The other arm of the ‘T’ (State 260) is in fluid communication with the vacuum system. A solenoid valve at the primary system inlet (S4) is used to prevent activation of an ENDS during a clean air puff. Modules and measurement instruments between State 2 and 490 can be added, removed, and interchanged. The number of bifurcated exposure chambers (BECs0), which are individually labelled with Greek letters, can be reduced to expanded. The vacuum systems have their own closed loop flow path that takes in room air and exhausts to an exhaust handling system, such as a building exhaust handling system.

### Additional Embodiments

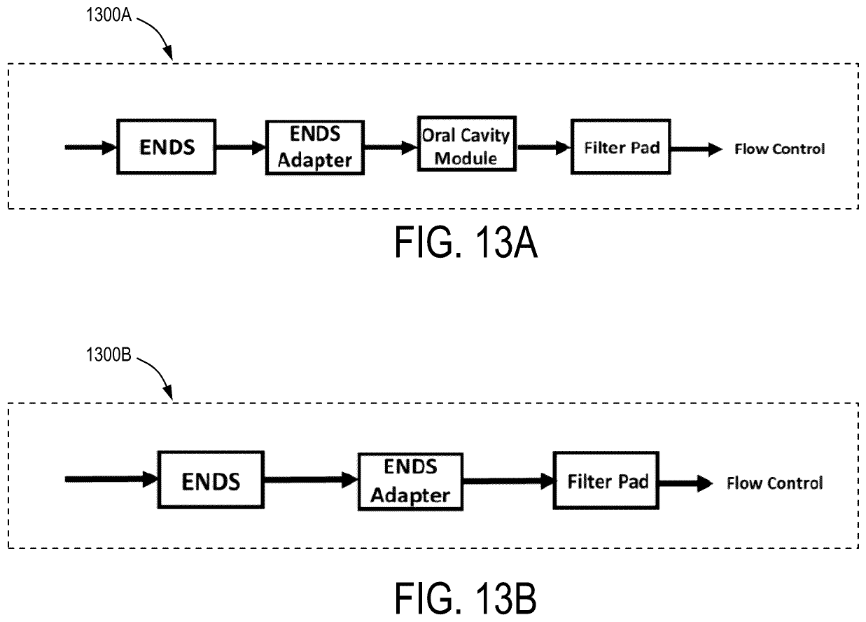

With reference to FIGS. 13A and 13B, two additional embodiments of a biomimetic aerosol exposure system 1300A, 1300B are illustrated in accordance with various aspects of the present disclosure. According to the present disclosure, each of these configurations can be utilized to study the effect of respiratory topography and sample location on mass distribution, among other features.

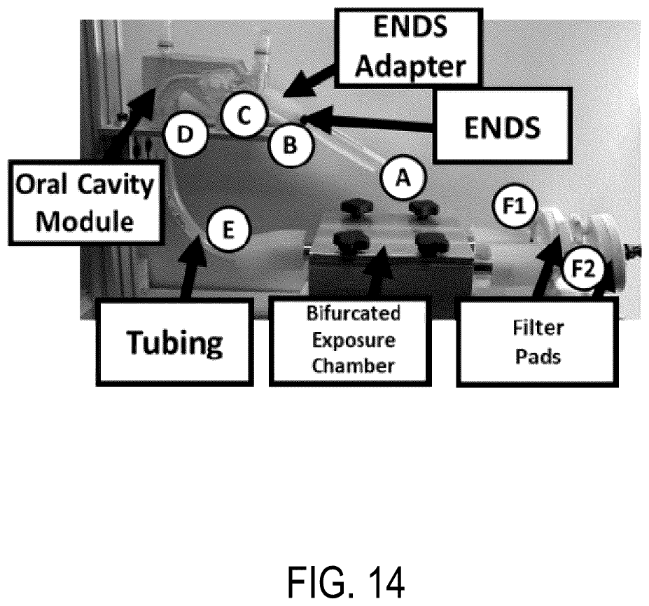

With reference to FIG. 14, an annotated photograph of a full assembly configuration of a biomimetic aerosol exposure system (e.g., biomimetic aerosol exposure system 100) is shown in accordance with various aspects of the present disclosure.

### Methods of Mimickinq Respiration Behavior

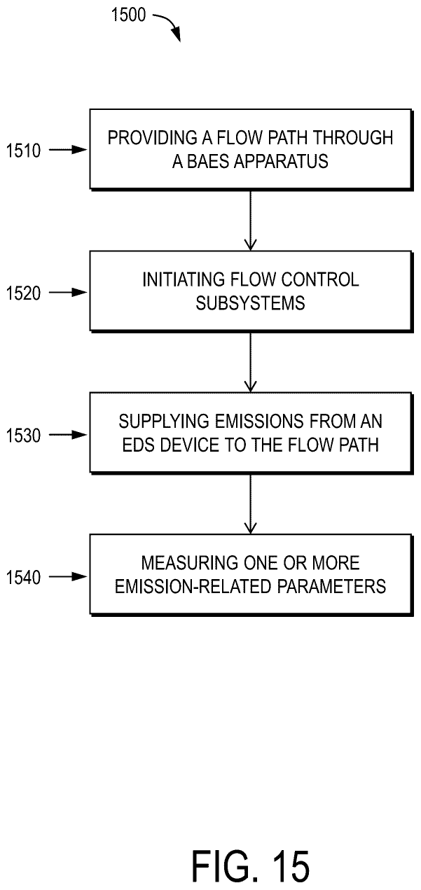

A Iso provided herein are methods of mimicking respiration behavior in humans for the purposes of performing emissions testing, toxicology testing, and/or similar studies. For example, with reference to FIG. 15, a flowchart illustrating a method 1500 for mimicking respiration behavior is shown in accordance with various aspects of the present disclosure. As shown, the method 1500 can include in at least a first step 1510, providing a flow path through a biomimetic aerosol exposure system (e.g., biomimetic aerosol exposure systems 100, 1200, 1300A, 1300B, etc.), wherein the biomimetic aerosol exposure system comprises the following modular subsystems: an emissions delivery system 102, an emissions device adapter 104, an oral cavity module 106, at least one bifurcated exposure chamber 110, and a flow control subsystem 114. In particular embodiments, the biomimetic aerosol exposure system (e.g., biomimetic aerosol exposure systems 100, 1200, 1300A, 1300B, etc.) may provide an extended flow path that includes one or more additional components, as described above.

The method 1500 may also include, in a step 1520, initiating the flow control subsystems 114 in order to simulate different behaviors of human respiration. For example, the following tables demonstrate the process logic for certain emissions topography profiles in connection with the biomimetic aerosol exposure system 1200 illustrated in FIG. 12.

TABLE 2
Process logic for an Emissions Topography Profile with discrete puffs. Values for
flowrate, puff duration, and post-puff gap are programmed by system operator.

| System | | | | | | | | | | |
|---|---|---|---|---|---|---|---|---|---|---|
| Flow | | Process | | | | | | | | |
| Condition | | Description | S1 | S2 | S3 | S4 | Q1 | Q2 | P1 | P2 |
| Discrete | | Prior to Puff | Open | Close | Close | Open | — | — | — | — |
| Puff | → | During Puff | Open | Close | Closed | Open | Measure | — | Modulate, | — |
| | \| | | | | | | at 901, | | signal | |
| | \| | | | | | | signal to | | from | |
| | \| | | | | | | controller | | controller | |
| | \| | End Puff | Close | Close | Close | Close | — | — | — | — |
| | \| | Post-Puff | Close | Close | Close | Close | — | — | — | — |
| | \| | Interval | | | | | | | | |
| | _{—} | Count Puffs | Close | Close | Close | Close | — | — | — | — |

TABLE 3
Process logic for an Emissions Topography Profile with discrete puffs followed
by inhales. Values for flowrate, puff duration, and post-puff gap, inhale
flowrate, and inhale duration are programmed by system operator.

| System | | | | | | | | | | |
|---|---|---|---|---|---|---|---|---|---|---|
| Flow | | Process | | | | | | | | |
| Condition | | Description | S1 | S2 | S3 | S4 | Q1 | Q2 | P1 | P2 |
| Discrete | | Prior to Puff | Open | Close | Close | Open | — | — | — | — |
| Puff | → | During Puff | Open | Close | Closed | Open | Measure | — | Modulate, | — |
| | \| | | | | | | at 901, | | signal | |
| | \| | | | | | | signal to | | from | |
| | \| | | | | | | controller | | controller | |
| | \| | End Puff | Close | Close | Close | Close | — | — | — | — |
| | \| | Post-Puff | Close | Close | Close | Close | — | — | — | — |
| | \| | Interval | | | | | | | | |
| | \| | Count Puffs | Close | Close | Close | Close | — | — | — | — |
| Nasal | \| | Prior to | Open | Open | Close | Close | — | — | — | — |
| Inhale | \| | Inhale | | | | | | | | |
| | \| | During | Open | Open | Close | Close | Measure | | Modulate, | |
| | \| | Inhale | | | | | at 901, | | signal | |
| | \| | | | | | | signal to | | from | |
| | \| | | | | | | controller | | controller | |
| | \| | End Inhale | Close | Close | Close | Close | — | — | — | — |
| | \| | Post-Inhale | Close | Close | Close | Close | — | — | — | — |
| | \| | Breath Hold | | | | | | | | |
| | _{—} | Count Cycle | Close | Close | Close | Close | — | — | — | — |

TABLE 4
Process logic for an Emissions Topography Profile with discrete puffs followed by inhales and exhales.
Values for flowrate, puff duration, and post-puff gap, inhale flowrate, and inhale duration, breath
hold, exhale flowrate, exhale duration, and inter-cycle gap are programmed by system operator.

| System | | | | | | | | | | |
|---|---|---|---|---|---|---|---|---|---|---|
| Flow | | Process | | | | | | | | |
| Condition | | Description | S1 | S2 | S3 | S4 | Q1 | Q2 | P1 | P2 |
| Discrete | | Prior to Puff | Open | Close | Close | Open | — | — | — | — |
| Puff | → | During Puff | Open | Close | Closed | Open | Measure | — | Modulate, | — |
| | \| | | | | | | at 901, | | signal | |
| | \| | | | | | | signal to | | from | |
| | \| | | | | | | controller | | controller | |
| | \| | End Puff | Close | Close | Close | Close | — | — | — | — |
| | \| | Post-Puff | Close | Close | Close | Close | — | — | — | — |
| | \| | Interval | | | | | | | | |
| | \| | Count Puffs | Close | Close | Close | Close | — | — | — | — |
| Nasal | \| | Prior to | Open | Open | Close | Close | — | — | — | — |
| Inhale | \| | Inhale | | | | | | | | |
| | \| | During | Open | Open | Close | Close | Measure | — | Modulate, | — |
| | \| | Inhale | | | | | at 901, | | signal | |
| | \| | | | | | | signal to | | from | |
| | \| | | | | | | controller | | controller | |
| | \| | End Inhale | Close | Close | Close | Close | — | — | — | — |
| | \| | Post-Inhale | Close | Close | Close | Close | — | — | — | — |
| | \| | Breath Hold | | | | | | | | |
| | \| | Count Cycle | Close | Close | Close | Close | — | — | — | — |
| Exhale | \| | Prior to | Close | Close | Open | Close | — | — | — | — |
| | \| | Exhale | | | | | | | | |
| | \| | During | Close | Close | Open | Close | — | Measure | — | Modulate, |
| | \| | Exhale | | | | | | at 280, | | signal |
| | \| | | | | | | | signal to | | from |
| | \| | | | | | | | controller | | controller |
| | \| | End Exhale | Close | Close | Close | Close | — | — | — | — |
| | \| | Post-Exhale | Close | Close | Close | Close | — | — | — | — |
| | \| | Breath Hold | | | | | | | | |
| | \| | Count PAR | Close | Close | Close | Close | — | — | — | — |
| | \| | Cycles | | | | | | | | |
| | \| | Count | Close | Close | Close | Close | — | — | — | — |
| | \| | Sedentary | | | | | | | | |
| | \| | Cycles | | | | | | | | |
| | \| | Count | Close | Close | Close | Close | — | — | — | — |
| | \| | Active | | | | | | | | |
| | _{—} | Cycles | | | | | | | | |

As described herein, the flow rates at which the EDS devices 102 are puffed can be small in comparison to the deep inhale users typically take following a puff or flows rates associated with tidal breathing. Tidal breathing flow rates are too high for most devices like e-cigarettes, excluding high powered box-mod devices. In the BAES 100 of the present disclosure, puffing is intended to generate aerosol from the EDS device 102 under realistic device usage conditions and the subsequent clean air inhalation taken is intended to maintain a physiological environment (e.g., shear stress, etc.) in the system. Therefore, inhalation flow rate may be maximized, subject to constraints on Reynolds number of wall shear stress.

In embodiments, global parameters may applied to the system as shown in the table below:

TABLE 5
Global parameters for a first principles model of
optimal inlet flow rate.

| Parameter | Value | Unit | Citation |
|---|---|---|---|
| fmin_{puff} | 0 | [Pa] | |
| fmax_{puff} | 2 | [Pa] | [172] |
| qmin_{puff} | 1.7 × 10⁻⁵ | [m3/s] | [85] |
| qmax_{puff} | 6.5 × 10⁻⁵ | [m3/s] | [85] |
| qminᵢₙₕₐₗₑ | 2.5 × 10⁻⁴ | [m3/s] | [97] |
| qmaxᵢₙₕₐₗₑ | 0.001 | [m3/s] | [97] |
| rho | 1.23 | $[ \frac{kg}{{m}^{3}} ]$ | |
| μ | 1.79 × 10⁻⁵ | [Pa * s] | |
| ReMax | 2100 | [−] | |

Additional emissions topography profiles have been developed based on clinically relevant puff and inhalation volumes, and are shown in the table below:

TABLE 5
Emissions topography profiles based on clinically relevant puff, puff associated respiration (PAR), and
tidal breathing volumes. Profiles that include clean air inhalation are named with their PAR flow rates.

| | | List of Topography | | Puff | Puff | PAR | PAR | PAR | PAR |
|---|---|---|---|---|---|---|---|---|---|
| | | Profiles | | Only | Only | 150 | 250 | 350 | 450 |
| System | Pre-Cleansing | Flow Rate | [mL/s] | 10 | 10 | 100 | 100 | 100 | 100 |
| Start-Up | Breath | Duration | [s] | 10 | 10 | 4 | 4 | 4 | 4 |
| Protocol | | Volume | [mL] | 100 | 100 | 400 | 400 | 400 | 400 |
| | | Gap | [s] | 10 | 10 | 20 | 20 | 20 | 20 |
| | | Repetitions | [—] | 2 | 2 | 1 | 1 | 1 | 1 |
| Puff and | Puff | Flow Rate | [mL/s] | 25 | 25 | 25 | 25 | 25 | 25 |
| Respiration | | Duration | [s] | 3.5 | 3.5 | 3.5 | 3.5 | 3.5 | 3.5 |
| Cycle (PRC) | | Volume | [mL] | 87.5 | 87.5 | 87.5 | 87.5 | 87.5 | 87.5 |
| | | Post-Puff Gap | [s] | 10 | 10 | 1 | 1 | 1 | 1 |
| | | Number of Puffs Per PRC | [—] | 1 | 1 | 1 | 1 | 1 | 1 |
| | Puff | Inhale Flow Rate | [mL/s] | 0 | 0 | 150 | 250 | 350 | 450 |
| | Associated | Inhale Duration | [s] | 0 | 0 | 10 | 6 | 4.29 | 3.33 |
| | Respiration (PAR) | Inhale Hold/Post-PAR Gap | [s] | 0 | 0 | 2 | 2 | 2 | 2 |
| | | Volume | [mL] | 0 | 0 | 1500 | 1500 | 1501.5 | 1498.5 |
| | | Exhale Flow Rate | [mL/s] | 0 | 0 | 0 | 0 | 0 | 0 |
| | | Exhale Duration | [s] | 0 | 0 | 0 | 0 | 0 | 0 |
| | | Exhale Hold | [s] | 0 | 0 | 0 | 0 | 0 | 0 |
| | | Number of PARs Per PRC | [—] | 0 | 0 | 1 | 1 | 1 | 1 |
| | Tidal | Flow Rate | [mL/s] | 0 | 0 | 150 | 250 | 350 | 450 |
| | Breathing | Inhale Duration | [s] | 0 | 0 | 3.33 | 2 | 1.43 | 3.33 |
| | | Inhale Hold/Post-Tidal Gap | [s] | 0 | 0 | 2 | 2 | 2 | 2 |
| | | Volume | [mL] | 0 | 0 | 499.5 | 500 | 500.5 | 1165.5 |
| | | Exhale Flow Rate | [mL/s] | 0 | 0 | 0 | 0 | 0 | 0 |
| | | Exhale Duration | [s] | 0 | 0 | 0 | 0 | 0 | 0 |
| | | Exhale Hold | [s] | 0 | 0 | 0 | 0 | 0 | 0 |
| | | Number of Tidal Breaths | [—] | 0 | 0 | 5 | 5 | 5 | 5 |
| | | Inter-PRC Gap | [s] | 0 | 0 | 3 | 3 | 3 | 3 |
| | | Number of PRCs | [—] | 50 | 25 | 25 | 25 | 25 | 25 |
| System Shut- | Post- | Flow Rate | [mL/s] | 10 | 10 | 0 | 0 | 0 | 0 |
| Down | Cleansing | Duration | [s] | 10 | 10 | 0 | 0 | 0 | 0 |
| Protocol | Breath | Volume | [mL] | 100 | 100 | 0 | 0 | 0 | 0 |

Next, in a step 1530, the method 1500 can include supplying emissions from an EDS device 102 to the flow path. In certain embodiments, this can include emissions from an e-cigarette. However, it is also contemplated that other emissions are provided, including but not limited to nicotine, cannabidiol (CBD), gases, solvents, chemicals, pathogens, and irritants.

With respect to steps 1520 and 1530, it should be appreciated that the flow control subsystems and the supply of the emissions may occur concurrently and/or independently in order to provide improved biomimicry. That is, these steps are not strictly sequential but may be performed and repeated as desired. For example, a typical experiment might study the effect of utilizing an e-cigarette over the course of a week or a month, and therefore the step 1530 may be repeated a number of times over a period of time, during which time clean air may also be introduced via the flow control subsystems. One or more of these subsystems may be connected with a controller comprising one or more computer processors and memory containing programmable instructions for performing the steps and methods described herein.

Next, in a step 1540, the method 1500 can include measuring one or more emissions-related parameters. This can occur at various phases of a study and these parameters can be measured at different stages of the modular biomimetic aerosol exposure system 100. For example, for inhaled tobacco products, the following doses may be reported:

| Symbol | Units | Description |
|---|---|---|
| Y_{TPM}″ | mg/cm² | Total particulate matter per |
| | | unit area |
| df_{TPM}″ | 1/cm² | Deposition fraction of TPM |
| | | per unit area |

These and other emissions-related testing outcomes can be determined by using the biomimetic aerosol exposure systems 100 of the present disclosure in accordance with the methods 1500 described herein, and further in accordance with the discussions in Sarles, Samantha Emma, “Biomimetic Aerosol Exposure System for in vitro Human Airway Exposure Studies” (2023). Thesis. Rochester Institute of Technology. Accessed from https://repository.rit.edu/theses/11577, the entire contents of which are incorporated herein by reference.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The terms “individual”, “patient”, and “subject” are used herein synonymously and interchangeably unless otherwise specified explicitly or implicitly by the context of its use. In various examples, these terms may refer to a human person, but can also refer to a non-human animal.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. M ultiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

A computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. M ore generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A biomimetic aerosol exposure system, the system comprising:
an emissions delivery system device comprising an emissions outlet and configured to generate at least a first type of emissions;
an oral cavity module comprising a biomimetic exposure chamber, an inlet, and an outlet, wherein the biomimetic exposure chamber mimics one or more parameters of a human oral cavity;
an emissions delivery system adapter configured to connect at least a portion of the emissions delivery system device including the emissions outlet at a first interface with the inlet of the oral cavity module at a second interface; and
a bifurcated exposure chamber operatively connected to the outlet of the oral cavity module.

2. The biomimetic aerosol exposure system of claim 1, wherein the emissions delivery system adapter, the oral cavity module, and the bifurcated exposure chamber create a flow path that mimics human respiration.

3. The biomimetic aerosol exposure system of claim 2, further comprising a flow control subsystem configured to control a flow of clean air and a flow of emissions from the emissions delivery system device throughout the flow path.

4. The biomimetic aerosol exposure system of claim 1, wherein the emissions delivery system adapter comprises an intermediate region between the first interface where the emissions delivery system device is received and the second interface where the inlet of the oral cavity module is received, and wherein the intermediate region provides a streamlined flow path configured to minimize mass loss of the emissions.

5. The biomimetic aerosol exposure system of claim 1, wherein the emissions delivery system adapter is formed from silicone rubber.

6. The biomimetic aerosol exposure system of claim 1, wherein oral cavity module further comprises one or more alternate inlets, at least one of which is located approximately at a region corresponding to a soft palate of a human.

7. The biomimetic aerosol exposure system of claim 1, wherein the biomimetic exposure chamber of the oral cavity module has an open volume of from about 24 cm³ to about 75 cm³, a surface area of from about 75 cm² to about 230 cm², and/or a L_{OCM} of from about 6 cm to about 20 cm.

8. The biomimetic aerosol exposure system of claim 1, wherein the bifurcated exposure chamber comprises an inlet, a first and a second outlet, and an enclosure having a lid defining an interior volume of the enclosure.

9. The biomimetic aerosol exposure system of claim 8, wherein the bifurcated exposure chamber further comprises a bifurcated flow path part defining a bifurcated flow channel, a multiple well plate, and one or more cell culture inserts disposed within the interior volume of the enclosure.

10. The biomimetic aerosol exposure system of claim 9, wherein the bifurcated flow path part is configured to lay on top of the multiple well plate within the enclosure and to guide at least the first type of emissions from the emissions delivery system device and through the bifurcated flow channel.

11. A bifurcated exposure chamber for use in a biomimetic aerosol exposure system, the bifurcated exposure chamber comprising:
an enclosure defining a first end, a second end, and an interior volume;
an inlet disposed at the first end and configured to be coupled to an inlet tubing; and
a first and a second outlet disposed at the second end and configured to be coupled to exit tubing.

12. The bifurcated exposure chamber of claim 11, wherein the first and second ends are arranged 180° from one another.

13. The bifurcated exposure chamber of claim 11, further comprising:
a bifurcated flow path part defining a bifurcated flow channel through the bifurcated flow path part, wherein the bifurcated flow path part is disposed or configured to be disposed within the interior volume of the enclosure.

14. The bifurcated exposure chamber of claim 13, wherein the bifurcated flow channel comprises a first channel in communication with the inlet and bifurcates into a second and a third channel in communication with the first and second outlets, respectively.

15. The bifurcated exposure chamber of claim 14, wherein an overall length of the bifurcated flow path through the bifurcated flow path part is from about 63 mm to about 200 mm, a length of the first channel before the bifurcation is from about 26 mm to about 80 mm, a width of the first channel before the bifurcation is from about 8 mm to about 25 mm, a height of the first channel before the bifurcation is from about 8 mm to about 25 mm, a length of each of the second and third channels is from about 36 mm to about 110 mm, a width of each of the second and third channels is from about 7 mm to about 22 mm, and/or a height of each of the second and third channels is from about 4 mm to about 15 mm.

16. The bifurcated exposure chamber of claim 14, wherein the bifurcated flow path has a branching angle of from about 30° to about 60°.

17. The bifurcated exposure chamber of claim 13, wherein the bifurcated flow path part comprises through-holes configured to receive cell culture inserts when assembled together with a multi-plate well within the interior volume of the enclosure.

18. The bifurcated exposure chamber of claim 17, further comprising:
the multi-plate well; and
one or more cell culture inserts having one or more cell culture exposure surfaces;
wherein the bifurcated flow path part is configured to lay on top of the multi-plate well within the enclosure and to guide aerosol flow tangentially across the one or more cell culture exposure surfaces within the bifurcated exposure chamber using physiologically relevant geometry and flow conditions.

19. A method for mimicking respiration behavior, the method comprising:
providing a flow path through a biomimetic aerosol exposure system, the biomimetic aerosol exposure system comprising:
an emissions delivery system device comprising an emissions outlet and configured to generate at least a first type of emissions;
an oral cavity module comprising a biomimetic exposure chamber, an inlet, and an outlet, wherein the biomimetic exposure chamber mimics one or more parameters of a human oral cavity;
an emissions delivery system adapter configured to connect at least a portion of the emissions delivery system device including the emissions outlet at a first interface with the inlet of the oral cavity module at a second interface; and
a bifurcated exposure chamber operatively connected to the outlet of the oral cavity module.

20. The method of claim 19, further comprising:
initiating a flow control subsystem of the biomimetic aerosol exposure system; and
supplying at least the first type of emissions from the emissions delivery system device to the flow path through the biomimetic aerosol exposure system.
